(19) United States
(12) Patent Application Publication
(10) Pub. No.: US 2002/0050276 A1
(43) Pub. Date: May 2, 2002

(54) METHOD AND BOTTLE FOR INFANT FEEDING

(76) Inventor: NIVA SHAPIRA, TEL AVIV (IL)

Correspondence Address: ATTEN: SOL SHEINBEIN, G.E. EHRLICH (1995) LTD., c/o ANTHONY CASTORINA, 2001 JEFFERSON DAVIS HIGHWAY, SUITE 207, ARLINGTON, VA, 22202, US

(21) Appl. No.: 09/341,266

(22) PCT Filed: Jan. 5, 1998
(86) PCT No.: PCT/US98/00915

(51) Int. Cl. A61B 19/00

(52) U.S. Cl. 128/898

(57) ABSTRACT

A method for feeding an infant by non-human milk including the steps of (a) feeding the infant by fore-milk equivalent having a volume of 30-60% of a total meal and a percentage of fat of 2.5-3.5%; and (b) feeding the infant by hind-milk equivalent having a volume of 40-70% of the total meal and a percentage of fat of 3.7-5.5%

### FIELD AND BACKGROUND OF THE INVENTION

The present invention relates to a method and bottle for infant feeding by non-human milk and, more particularly, to a method for infant feeding by non-human milk which mimics breast fore- and hind-milk feeding and a bottle for effecting the method.

Most researchers and authorities agree that under normal conditions exclusive breast feeding provides substantially an adequate amount of most nutrients for infants for at least four months. Breast fed infants have advantages in terms of general health. They have a more effective immune system and are therefore less sensitive to infections, such as but not limited to, ears, lungs and intestinal infections, they achieve faster mental development and their body composition comprises less fat. Also, there are indications that breast feeding achieves better future health of the child and the adult. For example, breast fed children are less prone to develop juvenile diabetes, heart diseases, obesity, etc. For further advantages of breast feeding the reader is referred to Am. J. Clin. Nutr. 40:635-646, 1984, which is incorporated by reference as if fully set forth herein.

It has been found that in the course of the breast feeding the infant is fed initially with milk having a smaller percentage of fat, which is known in the art as fore-milk, and thereafter with milk having a larger percentage of fat, which is known in the art as hind-milk. To this effect the reader is referred to J. of Ped. and Gastro. and Nutr. 18:474-477, 1994, which is incorporated by reference as if fully set forth herein.

In attempts to become as close as possible to breast feeding, formula producers invest a lot of effort in improving the composition of so called “humanised milk”. It has been found that formula fed infants tend to eat more calories and protein and to grow faster than breast fed infants. However, whereas in the past the rate of growth was perceived as an indication of good nutritional status. in recent years it became clear that facilitated growth is not necessarily advantageous to the present and future health of the child. To this effect the reader is referred to Am. J. Clin. Nutr. 58:152-161, 1993, and J. of Ped. and Gastro. and Nutr. 18:474-477. 1994. which are incorporated by reference as if fully set forth herein.

In view of the above research, it has become desirable to develop a method of feeding infants that provides a gradual increase of “fat content” which is similar to that of the breast feeding.

The advantage of the gradual method is assumed to be, inter alia, better compatibility between the infant's requirements and consumption. Infants, who consume mostly the fore-milk which is lower in fat content, will get more of the high carbohydrate low protein formula as the result of which they will get more protein which is spared (by carbohydrates) mainly for true growth of the infant, e.g. body length, without being satisfied for prolonged times and without over consumption of calories from fat. Bigger and stronger babies who spend and require more energy, will get at the beginning more carbohydrate and protein, and later on more calories, but not as much protein, from extra suckling, as the fat content of the formula increases along the feeding session in, for example, a logarithmic or geometric manner.

There is thus a widely recognized need for, and it would be highly advantageous to have, a method for infant feeding by non-human milk which mimics breast fore- and hind-milk feeding, and a bottle for effecting the method.

### SUMMARY OF THE INVENTION

According to the present invention there is provided a method for feeding an infant by non-human milk.

According to further features in preferred embodiments of the invention described below, the method comprising the steps of (a) feeding the infant by fore-milk equivalent having a volume of, for example, 30-60% of a total meal and a percentage of fat of, for example, 2.5-3.5%; and (b) feeding the infant by hind-milk equivalent having a volume of, for example, 40-70% of the total meal and a percentage of fat of, for example, 3.7- 5.5%.

According to still further features in the described preferred embodiments the feeding is performed either by feeding the fore-milk equivalent and the hind-milk equivalent one after another or by first feeding the fore-milk equivalent which is gradually admixed with the hind-milk equivalent.

According to still further features in the described preferred embodiments the percentage of the fore-milk equivalent in the total meal volume is 31-47% and the fat content therein is 2.8% -3.3%, and the percentage of the hind-milk equivalent in the total meal volume is 53-67% and the fat content therein is 4.5%- 5%.

According to still further features in the described preferred embodiments the percentage of the fore-milk equivalent in the total meal is 40% and the fat content therein is 3%, and the percentage of the hind-milk equivalent in the total meat volume is 60% and the fat content therein is 4.8%.

According to still further features in the described preferred embodiments the feeding is performed in two bottles one being filled with the fore-milk equivalent and one being filled with the hind-milk equivalent, the contents of the bottles being forwarded one after the other.

According to still further features in the described preferred embodiments the feeding is performed with a bottle which is divided into two compartments, one being filled with the fore-milk equivalent and the other with the hind-milk equivalent, the bottle being designed in such a manner enabling the infant to consume at first the fore-milk equivalent and thereafter the fore-milk equivalent gradually admixed with the hind-milk equivalent.

According to another embodiment provided is a bottle for feeding an infant, which is suitable for effecting the feeding method herein described.

According to still further features in the described preferred embodiments the bottle comprising first and second compartments and being designed in such a manner enabling the infant to consume at first a content being held in the first compartment and thereafter admixing same with a content being held in the second compartment.

The present invention successfully addresses the shortcomings of the presently known configurations by providing a method and bottle for infant feeding by a non-human milk which mimics breast fore- and hind-milk feeding.

### BRIEF DESCRIPTION OF THE DRAWINGS

The invention herein described, by way of example only, with reference to the accompanying drawings, wherein:

FIG. 1 shows a front view of a bottle according to the present invention; and

FIG. 2 shows a front view of another embodiment of a bottle according to the present invention.

### DESCRIPTION OF THE PREFERRED EMBODIMENTS

The present invention is of a method for infant feeding by non-human milk and of a bottle for effecting the method, which can be used for mimicking breast fore- and hind-milk feeding. Specifically, the present invention can be used to gradually increase the fat content fed to the infant along a feeding session and thereby to mimic the natural case in which low fat fore milk followed by higher fat hind milk are fed to the infant.

The principles and operation of a method and bottle according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Thus, the present invention is of a method for gradual feeding of infants by non-human milk “infant formula”, wherein milk (fore-milk equivalent) having a volume of, for example, 30-60% of a total meal and a percentage of fat of, for example, 2.5-3.5%, and milk (hind-milk equivalent) having a volume of, for example, 40-70% of the total meal and a percentage of fat of, for example, 3.7-5.5% are fed to the infant. A meal may have a total volume of, for example, 30-300 ml. depending on infant's intake, age, weight, etc.

The term “milk equivalent” a used herein in the specification and in the claims section below refers to any human milk substitute used to feed infants. The substitute may be milk based or non-milk based (e.g., soy based). The terms “fore-milk equivalent” and “hind-milk equivalent” therefore refer to milk substitutes, as this term defined herein, which are similar or compatible at least in their fat content and/or other ingredients to human fore and hind milk, respectively.

According to the present invention feeding is performed either by feeding fore-milk equivalent and hind-milk equivalent one after another or alternatively by first feeding fore-milk equivalent which is gradually admixed with the hind-milk equivalent.

The percentage of the fore-milk equivalent in the total meal volume is 31-47%, advantageously 40%; and the fat content therein is preferably 2.8-3.3%, advantageously 3%.

The percentage of the hind-milk equivalent in the total meal volume is preferably, 53-67%, advantageously 60%; and the fat content therein is preferably 4.5% -5%, advantageously 4.8%.

The several formulas to be fed by the method according to the present invention may be prepared, e.g.. by dissolving in water a dry powder having the adequate formulation to achieve the correct content. The dissolving operation may be made either directly in the bottle or in a factory or the like where a large quantity of formulations is prepared and/or marketed as ready to feed liquid formula.

Milk base formulations according to the present invention may include the following ingredients: lactose, vegetable oils, skimmed milk powder, whey protein concentrate, Sodium, Calcium, Phosphorus, Potassium, Chloride, Iron, Magnesium, Taurine and Vitamins.

Soy base formulations according to the present invention may include the following ingredients: Glucose syrup, vegetable oils, soy protein isolate, Sucrose, Maltodextrine, Sodium, Calcium, Phosphorus, Potassium, chloride, Iron, Magnesium, Vitamins, Methionine, Taurine, Carnitine, and trace element.

Detailed composition of presently prefered so, and milk-based formulations of fore- and hind-milk equivalents are given in the Examples section hereinbelow.

The method according to the present invention may be performed by feeding with two separate bottles or a dual chamber bottle (as described, for example, in U.S. Pat. Nos. 5,593,052, 5,611,776, 5,419,445; 4,971,211, which are incorporated by reference as if fully set forth herein). i.e., by feeding fore-milk equivalent and hind-milk equivalent separately one after another, or with one combination bottle enabling the gradual admixing of hind-milk equivalent into fore-milk equivalent.

When the method is performed with two bottles (or a dual chamber bottle) each bottle (or chamber) is filled with one of the formulations. In this case at first the bottle (or chamber) containing the fore-milk equivalent is fed and thereafter the bottle (or chamber) containing the hind-milk equivalent. By this method the bottles utilised are conventional bottles for feeding infants.

Preferably, the method is performed by feeding, with one combination bottle which is divided into two compartments, one being filled with fore-milk equivalent and the second one with hind-milk equivalent, the bottle being, designed in such a manner enabling the infant to consume at first the fore-milk equivalent which is thereafter gradually admixed with the hind-milk equivalent.

The combination bottles which enable the feeding of infants by the method claimed and described herein are also within the scope of the present invention.

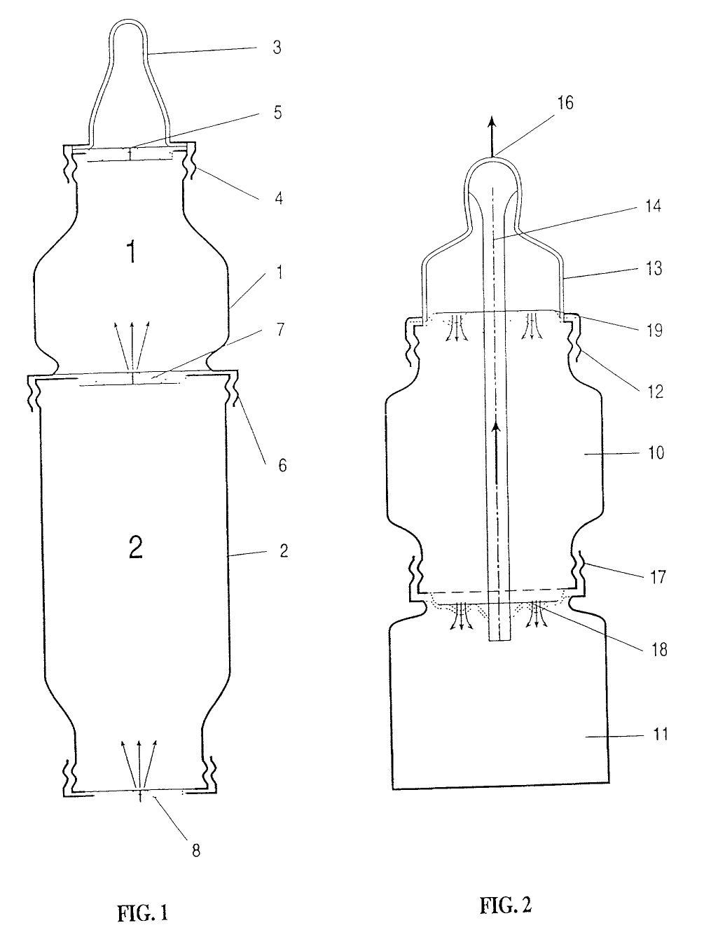

The bottle shown in FIG. 1 includes two compartments, compartment 1 for fore-milk equivalent and compartment 2 for hind-milk equivalent. Compartment 1 is connected at the front end to teat 3 by thread 4 and by non return diaphragm valve 5. At its back end compartment 1 is connected to the front end of compartment 2 by thread 6 and by non return diaphragm valve 7. The back end of compartment 2 is provided with non return diaphragm valve 8 which enables air to flow into compartment 2. Valves 8, 7 and 5 enable the flow of air and/or milk in the same direction, i.e. from the back end of compartment 2 to compartment 1 and to teat 3. Thus, the moment the infant starts to suckle, milk is flowing to its mouth, at the beginning only the fore-milk equivalent and thereafter a gradual mixture of same with the hind-milk equivalent.

The bottle shown in FIG. 2 includes also two compartments, compartment 10 for hind-milk equivalent and compartment 11 for fore-milk equivalent. Thread 12 connects the front end of compartment 10 to teat 13. Teat 13 is in fluid connection by straw stem 14 to compartment 11. Hole 16 in teat 13 enables free flow of atmospheric air to compartment 10 via non return diaphragm valve 19. The hack end of compartment 10 is connected to the front end of compartment 11 by thread 17 and non return diaphragm valve 18. The moment the infant starts to suckle via teat 13 and straw stem 14, liquid flows from compartment 11 and as a result of the vacuum, air flows through hole 16 to compartment 10, pushing at the beginning pure fore-milk equivalent from compartment 11 through straw stem 14 and thereafter a gradual mixture thereof with the hind-milk equivalent flowing from compartment 10 to compartment 11 and from there via straw stem 14 to the mouth of the infant.

The present invention will now, be illustrated with reference to the following examples and to the accompanying drawings without being restricted by them.

### EXAMPLE 1

### Infant Formula I (Milk Base)

The formulation comprises the following ingredients: lactose, vegetable oils, skimmed milk powder, whey protein concentrate, Sodium, Calcium, Phosphorus, Potassium, Chloride, Iron, Magnesium, Taurine and Vitamins. A special formulation is given hereunder:

| | | Powder | Regular | Fore-Milk Eq. | Hind-Milk Eq. |
|---|---|---|---|---|---|
| | Unit | 100 g | 100 ml | 100 ml | 100 ml |
| General Comp. | | | | | |
| Protein | gram | 11 1 | 1 5 | 1 5 | 1.5 |
| Fat | gram | 25.9 | 3.5 | 2.9 | 4.8 |
| Lactose | gram | 55 5 | 7 5 | 7 5 | 7 5 |
| Water | gram | 2 5 | — | — | — |
| Ash | gram | 2 06 | 0 27 | 0.27 | 0.27 |
| Vitamins | | | | | |
| Vitamin A | I.U. | 1500 | 200 | 200 | 200 |
| Vitamin D | I.U. | 300 | 40 | 40 | 40 |
| Vitamin E | mg | 6 | 0.81 | 0.81 | 0.81 |
| Vitamin K | μg | 15 | 2.01 | 2.01 | 2.01 |
| Vitamin B1 | μg | 350 | 47.03 | 47.03 | 47.03 |
| Vitamin B2 | μg | 450 | 60 | 60 | 60 |
| Vitamin B6 | μg | 222 | 30 | 30 | 30 |
| Vitamin B12 | μg | 0.66 | 0.09 | 0.09 | 0.09 |
| Niacin | mg | 2 | 0.27 | 0.27 | 0.27 |
| Folic Acid | μg | 45 | 6 | 6 | 6 |
| Calcium | mg | 4.44 | 0.06 | 0.06 | 0.06 |
| Pantothenate | | | | | |
| Biotin | μg | 11 | 1.5 | 1.5 | 1.5 |
| Vitamin C | mg | 45 | 6 08 | 6.08 | 6 08 |
| Minerals | | | | | |
| Calcium | mg | 326 | 44 | 44 | 44 |
| Phosphorus | mg | 219 | 29 6 | 29 6 | 29 6 |
| Magnesium | mg | 37 | 5 | 5 | 5 |
| Iron | mg | 7.4 | 1 | 1 | 1 |
| Sodium | mg | 120.7 | 16.3 | 16.3 | 16.3 |
| Potassium | mg | 373 | 50 | 50 | 50 |
| Ca/p ratio | — | 1.49 | 1.49 | 1.49 | 1.49 |
| Amino Acid Profile | | | | | |
| Alanine | mg | 522 | 69.6 | 69.6 | 69.6 |
| Arginine | mg | 368 | 49.1 | 49.1 | 49.1 |
| Asparatic Acid | mg | 11.10 | 1.5 | 1.5 | 1.5 |
| Cystine | mg | 191 | 25.5 | 25.5 | 25.5 |
| Glutamic Acid | mg | 1423 | 189.7 | 189.7 | 189.7 |
| Glycine | mg | 244 | 32.5 | 32.5 | 32.5 |
| Histidine | mg | 262 | 34.9 | 34.9 | 34.9 |
| Isoleucine | mg | 761 | 101.5 | 101.5 | 101.5 |
| Leucine | mg | 12.20 | 1.62 | 1.62 | 1.62 |
| Lysine | mg | 10.00 | 1.3 | 1.3 | 1.3 |
| Methionine | mg | 270 | 36 | 36 | 36 |
| Phenylalanine | mg | 461 | 62.3 | 62.3 | 62.3 |
| Proline | mg | 962 | 128.3 | 128.3 | 128.3 |
| Serine | mg | 681 | 90.8 | 90.8 | 90.8 |
| Taurine | mg | 37 | 4.9 | 4.9 | 4.9 |
| Threonine | mg | 686 | 91.5 | 91.5 | 91.5 |
| Tryptophan | mg | 180 | 24 | 24 | 24 |
| Tryosine | mg | 463 | 61 7 | 61 7 | 61.7 |
| Valine | mg | 775 | 103.3 | 103 3 | 103.3 |
| Fatty Acid Profile | | | | | |
| Caprylic (C8) | % from fa | 2.6 | 2.6 | 2.6 | 2.6 |
| Capric (C10) | % | 2 1 | 2.1 | 2.1 | 2.1 |
| Lauric (C12) | % | 17.5 | 17.5 | 17.5 | 17.5 |
| Meristic (C14) | % | 6.7 | 6 7 | 6 7 | 6.7 |
| Palmitic (C16) | % | 11.2 | 11.2 | 11.2 | 11.2 |
| Stearic (C18) | % | 11.8 | 11 8 | 11 8 | 11.8 |
| Oleic (C18:1) | % | 37.0 | 37.0 | 37.0 | 37.0 |
| Linoleic (C18:2) | % | 10.0 | 10.0 | 10.0 | 10.0 |
| Linolenic (C18.3) | % | 1.2 | 1.2 | 1 2 | 1.2 |

The powder described herein is suitable for the preparation of the regular liquid formula. Similar powders having less or more fat content (see row 2) may be used for the preparation of the fore- and hind-milk equivalents, respectively.

### EXAMPLE 2

### Infant Formula II (Soy Base)

The formulation comprises the following ingredients: Glucose syrup, vegetable oils, soy protein isolate, Sucrose, Maltodextrine, Sodium, Calcium, Phosphorus, Potassium, chloride, Iron, Magnesium, Vitamins, Methionine, Taurine, Carnitine, and trace element. A special formulation is given hereunder:

| | | Powder | Regular | Fore-Milk Eq. | Hind-Milk Eq. |
|---|---|---|---|---|---|
| | Unit | 100 g | 100 ml | 100 ml | 100 ml |
| General Comp. | | | | | |
| Protein | gram | 15 | 1.98 | 1.98 | 1.98 |
| Fat | gram | 27.54 | 3.64 | 3.0 | 4.7 |
| Carbohydrate | gram | 51.5 | 6.8 | 6.8 | 6.8 |
| Linoleic Acid | gram | 4.5 | 0.6 | 0 6 | 0.6 |
| Vitamins | | | | | |
| Vitamin A | I.U. | 1500 | 198 | 198 | 198 |
| Vitamin D | I.U. | 300 | 39 7 | 39 7 | 39.7 |
| Vitamin E | I.U. | 10 | 1.32 | 1.32 | 1.32 |
| Vitamin C | mg | 65 | 8.6 | 8.6 | 8.6 |
| Vitamin K | μg | 77 | 10.2 | 10.2 | 10.2 |
| Vitamin B1 | μg | 345 | 45.6 | 45.6 | 45.6 |
| Vitamin B2 | μg | 445 | 58 9 | 58 9 | 58.9 |
| Vitamin B6 | μg | 327 | 43.3 | 43.3 | 43.3 |
| Vitamin B12 | μg | 1.5 | 0.2 | 0 2 | 0.2 |
| Niacin | mg | 7 | 0.93 | 0 93 | 0.93 |
| Folic Acid | μg | 76 | 10 | 10 | 10 |
| Pantothenic Acid | μg | 4 5 | 0.6 | 0 6 | 0 6 |
| Biotin | μg | 25 | 3 3 | 3 3 | 3 3 |
| Choline | mg | 58 | 7.7 | 7 7 | 7 7 |
| Minerals | | | | | |
| Calcium | mg | 500 | 66.2 | 66.2 | 66.2 |
| Phosphorus | mg | 300 | 39.7 | 39.7 | 39 7 |
| Magnesium | mg | 45 | 6 | 6 | 6 |
| Iron | mg | 9.2 | 1.2 | 1.2 | 1.2 |
| Zinc | mg | 4 | 0.53 | 0.53 | 0.53 |
| Manganese | μg | 150 | 19.8 | 19.8 | 19.8 |
| Copper | μg | 400 | 53 | 53 | 53 |
| Iodine | μg | 77 | 10.2 | 10.2 | 10.2 |
| Sodium | mg | 200 | 26.5 | 26.5 | 26.5 |
| Potassium | mg | 546 | 72.2 | 72.2 | 72.2 |
| Chloride | mg | 400 | 53 | 53 | 53 |
| Inositol | mg | 25 | 3.3 | 3.3 | 3.3 |
| Caimtine | mg | 10 | 1.3 | 1 3 | 1 3 |
| Ca/P ratio | | 1.67 | 1 67 | 1 67 | 1.67 |
| Amino Acid Profile | | | | | |
| Alanine | mg | 640 | 85 3 | 85 3 | 85 3 |
| Arginine | mg | 497 | 6.5 | 6.5 | 6.5 |
| Aspartic Acid | mg | 1385 | 184 7 | 184.7 | 184 7 |
| Cystine | mg | 242 | 32 3 | 32.3 | 32.3 |
| Glutamic Acid | mg | 3065 | 408 7 | 408 7 | 408 7 |
| Glycine | mg | 300 | 40 | 40 | 40 |
| Histidine | mg | 382 | 50.9 | 50.9 | 50.9 |
| Isoleucine | mg | 893 | 119.1 | 119.1 | 119.1 |
| Leucine | mg | 1600 | 213.3 | 213.3 | 213.3 |
| Lysine | mg | 1360 | 181.3 | 181.3 | 181.3 |
| Methionine | mg | 406 | 54.1 | 54.1 | 54.1 |
| Phenylalanine | mg | 650 | 86.7 | 86.7 | 86.7 |
| Proline | mg | 1113 | 148 4 | 148.4 | 148.4 |
| Serine | mg | 737 | 98.3 | 98.3 | 98.3 |
| Taurine | mg | 51 | 6.8 | 6.8 | 6.8 |
| Threonine | mg | 460 | 61.3 | 61.3 | 61.3 |
| Tyrosine | mg | 621 | 82.8 | 82.8 | 82.8 |
| Valine | mg | 947 | 126.3 | 126.3 | 126.3 |
| Fatty Acid Profile | | | | | |
| Caprylic (C8) | % from fa | 2.6 | 2.6 | 2.6 | 2 6 |
| Capric (C10) | % | 2.1 | 2.1 | 2.1 | 2.1 |
| Lauric (C12) | % | 17.5 | 17.5 | 17 5 | 17.5 |
| Meristic (C14) | % | 6.7 | 6.7 | 6.7 | 6.7 |
| Palmitic (C16) | % | 11.2 | 11.2 | 11.2 | 11.2 |
| Stearic (C18) | % | 11.8 | 11 8 | 11.8 | 11.8 |
| Oleic (C18-1) | % | 37.0 | 37 0 | 37 0 | 37.0 |
| Linoleic (C18:2) | % | 10.0 | 10.0 | 10 0 | 10 0 |
| Linolenic (C18:3) | % | 1 2 | 1 2 | 1.2 | 1 2 |

The powder described herein is suitable for the preparation of the regular liquid formula. Similar powders having less or more fat content (see row 2) may be used for the preparation of the fore- and hind-milk equivalents, respectively.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method for feeding an infant by non-human milk, the method comprising the steps of
(a) feeding the infant by fore-milk equivalent; and
(b) feeding the infant by hind-milk equivalent.

2. The method of claim 1, wherein said fore-milk equivalent has a volume of 30-60% of a total meal fed to the infant and a percentage of fat of 2.5-3.5%, and said hind-milk equivalent has a volume of 40-70% of the total meal and a percentage of fat of 3.7-5.5%.

3. The method of claim 1, wherein said feeding is performed either by feeding said fore-milk equivalent and said hind-milk equivalent one after another or by first feeding said fore-milk equivalent which is gradually admixed with said hind-milk equivalent.

4. The method of claim 2, wherein the percentage of said fore-milk equivalent in the total meal volume is 31-47% and the fat content therein is 2.8%-3.3% and the percentage of said hind-milk equivalent in the total meal volume is 53-67% and the fat content therein is 4.5%-5%.

5. The method of claim 4, wherein the percentage of said fore-milk equivalent in the total meal is 40% and the fat content therein is 3%, and the percentage of said hind-milk equivalent in the total meal volume is 60% and the content therein is 4.8%.

6. The method of claim 1, wherein said feeding is performed using two bottles, one being filled with said fore-milk equivalent and one being filled with said hind-milk equivalent, the contents of said bottles being forwarded one after the other.

7. The method of claim 1, wherein said feeding is performed with a bottle which is divided into two compartments, one being filled with said fore-milk equivalent and the other with said hind-milk equivalent, the bottle being designed in such a manner enabling the infant to consume at first said fore-milk equivalent and thereafter said fore-milk equivalent gradually admixed with said hind-milk equivalent.

8. A bottle for feeding infants comprising first and second compartments and being designed in such a manner enabling the infant to consume at first a content being held in said first compartment and thereafter admixing same with a content being held in said second compartment.
